(19) United States
(12) Patent Application Publication
(10) Pub. No.: US 2007/0253921 A1
(43) Pub. Date: Nov. 1, 2007

(54) Composition comprising at least one cyanoacrylate monomer, at least one glucide compound and at least one liquid organic solvent, and cosmetic treatment processes using it

(76) Inventors: Gabin Vic, Venette (FR); Aude Livoreil, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/783,925

(22) Filed: Apr. 13, 2007

Related U.S. Application Data
(60) Provisional application No. 60/796,873, filed on May 3, 2006.

(30) Foreign Application Priority Data
Apr. 13, 2006 (FR) 0603229

(51) Int. Cl. A61K 8/81; A61K 8/73

(52) U.S. Cl. 424/63; 424/70.13; 424/70.16; 424/70.7

(57) ABSTRACT

The present disclosure relates to a cosmetic composition comprising at least one cyanoacrylate monomer, at least one glucide compound and at least one liquid organic solvent. The present disclosure also relates to a process for treating for and caring for keratinous fibers that comprises applying to the fibers the disclosed composition. The present disclosure also relates to a method for conferring a conditioning effect and/or a styling effect on keratinous fibers, such as the hair, by applying the disclosed composition thereto.

This application claims benefit of U.S. Provisional Application No. 60/796,873, filed May 3, 2006, the contents of which are incorporated herein by reference. This application also claims benefit of priority under 35 U.S.C. § 119 to French Patent Application No. FR 06/03329, filed Apr. 13, 2006, the contents of which are also incorporated herein by reference.

The present disclosure relates to a composition comprising at least one cyanoacrylate monomer, at least one glucide compound and at least one organic solvent, to its use in the cosmetic treatment of keratinous fibers and to a cosmetic treatment process employing such a composition.

In the field of cosmetics, one aim is to modify the surface properties of keratinous fibers, such as the hair, for example in order to contribute to the hair a conditioning effect, such as softness or gloss, or a styling effect which makes it possible to contribute body, bulk or volume to the hair.

To do this, use is generally made of cosmetic compositions based on conditioning agents, such as silicones or polymers, having a strong affinity for keratinous fibers such as the hair.

However, these conditioning or styling agents have a tendency to be removed during washing with shampoos, making it necessary to repeat the applications of the compositions to the hair.

In order to increase the persistence of the coating of polymers, it is possible to envisage carrying out a radical polymerization of certain monomers directly on the hair. However, strong damage to the hair fibers, probably related to the polymerization initiators, is observed. Thus, the hair treated is difficult to disentangle. The document FR 2 833 489 describes the polymerization of an electrophilic monomer on the hair.

Further, these sheathings are disrupted by the sebum produced by the sebaceous glands.

For instance, the hair becomes greasy again very rapidly over time and the hair then has a greasy and dirty appearance.

At the current date, there do not exist cosmetic compositions which make it possible to contribute body, bulk or volume in a lasting fashion to the hair while having good cosmetic properties over time.

The inventors have unexpectedly and surprisingly discovered that it is possible to obtain an improvement in the body, bulk or volume of the hair and a reduction in the regressing of the hair, this being achieved in a lasting fashion, by combining at least one glucide compound defined below, at least one cyanoacrylate monomer, and at least one organic solvent.

The properties described herein occur, at least in part, because a composition comprising such a combination results in the formation of a coating or sheathing which is persistent, for instance with respect to shampooing, even after several shampoo washings. The sheathing obtained can, in addition to the persistent styling properties, contribute properties of gloss and of conditioning.

The addition of a polysaccharide greatly reduces the effect due to the sebum. The hair becomes greasy again less rapidly over time while retaining good styling properties.

Surprisingly, the individual hairs remain completely separate and can be styled without problems.

Without being restricted to this explanation, it appears that it is the hydroxide ions present in the water absorbed by the hair which set in motion the process of anionic polymerization at the treatment composition/hair interface. The polymer thus formed in situ by interfacial anionic polymerization exists in the form of a homogeneous and smooth deposited layer and has excellent adhesion to hair.

One aspect of the present disclosure is a cosmetic composition comprising:
at least one cyanoacrylate monomer,
at least one glucide compound, and
at least one liquid organic solvent.

Another aspect of the present disclosure is the use of the composition in the cosmetic treatment of keratinous fibers, such as the hair.

A further aspect of the present disclosure is a process for the cosmetic treatment of the hair employing the composition.

Other subject matter, characteristics, aspects, advantages, and beneficial properties of the present disclosure will become even more clearly apparent upon reading the description and examples which follow.

According to at least one embodiment, the at least one liquid organic solvent is other than the glucide compound and the cyanoacrylate monomer.

The compositions of the present disclosure comprise at least one liquid organic solvent.

The at least one liquid organic solvent may be chosen from compounds which are liquid at a temperature of 25° C. and at 105 Pa (760 mmHg).

As used herein, the term “organic solvent” is understood to mean a substance capable of dissolving another substance without chemically modifying it.

The organic solvent is chosen, for example, from: aromatic alcohols, such as benzyl alcohol; liquid fatty alcohols, such as C₁₀-C₃₀ fatty alcohols; modified or unmodified polyols, such as glycerol, glycol, propylene glycol, dipropylene glycol, butylene glycol or butyl diglycol; volatile silicones, such as cyclopentasiloxane, cyclohexasiloxane, polydimethylsiloxanes modified or unmodified by alkyl and/or amine and/or imine and/or fluoroalkyl and/or carboxyl and/or betaine and/or quaternary ammonium functional groups; liquid modified polydimethylsiloxanes; mineral, synthetic organic or vegetable oils; alkanes, for instance C₅ to C₁₀ alkanes; liquid fatty acids; liquid fatty esters and further, for example, liquid fatty alcohol benzoates or salicylates; and their mixtures.

The organic solvent may be chosen from organic oils; silicones, such as volatile silicones, silicone gums or oils which may or may not be aminated and their mixtures; mineral oils; vegetable oils, such as olive oil, castor oil, rapeseed oil, coconut oil, wheat germ oil, sweet almond oil, avocado oil, macadamia oil, apricot oil, safflower oil, candlenut oil, camelina oil, tamanu oil or lemon oil; or also organic compounds, such as C₅-C₁₀ alkanes, acetone, methyl ethyl ketone, liquid esters of C₁-C₂₀ acids and of C₁-C₈ alcohols, such as methyl acetate, butyl acetate, ethyl acetate and isopropyl myristate, dimethoxyethane, diethoxyethane, liquid C₁₀-C₃₀ fatty alcohols, such as oleyl alcohol, liquid C₁₀-C₃₀ fatty alcohol esters, such as C₁₀-C₃₀ fatty alcohol benzoates, isononyl isononanoate, isostearyl malate, pentaerythrityl tetraisostearate, tridecyl trimelate and their mixtures; polybutene oil, the cyclopentasiloxane (14.7% by weight)/polydimethylsiloxane dihydroxylated in the α and ω positions (85.3% by weight) mixture, and their mixtures.

According to at least one embodiment, the organic solvent may comprise a silicone or a mixture of silicones, such as liquid polydimethylsiloxanes and liquid modified polydimethylsiloxanes, the viscosity of the silicone and/or of the mixture of silicones at 25° C. ranging from 0.1 cSt to 1,000,000 cSt, for example ranging from 1 cSt to 30 000 cSt.

Non-limiting mention may be made of the following silicones:

the α,ω-dihydroxylated polydimethylsiloxane/cyclopentadimethylsiloxane (14.7/85.3) mixture sold by Dow Corning under the name of DC 1501 Fluid;

the α,ω-dihydroxylated polydimethylsiloxane/polydimethylsiloxane mixture sold by Dow Corning under the name of DC 1503 Fluid;

the dimethicone/cyclopentadimethylsiloxane mixture sold by Dow Corning under the name of DC 1411 Fluid or that sold by Bayer under the name SF1214;

the cyclopentadimethylsiloxane sold by Dow Corning under the name of DC245 Fluid;

and the respective mixtures of these oils.

In addition to the liquid organic solvent or solvents, the compositions according to the present disclosure may comprise water. For instance, the compositions are anhydrous compositions, that is to say that they comprise less than 1% by weight of water, with respect to the total weight of the composition.

As disclosed herein, the at least one organic solvent is generally present in an amount ranging from 0.01 to 99% by weight, for instance from 0.1 to 50% by weight, with respect to the total weight of the composition.

According to at least one embodiment of the present disclosure, the at least one cyanoacrylate monomer of the present disclosure is chosen from the monomers of formula (I): 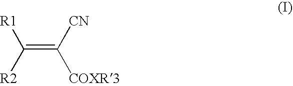
wherein:
X is chosen from NH, S and O,
R1 and R2 each denote hydrogen atoms,
R′3 is chosen from a hydrogen atom, a saturated, unsaturated, linear, branched or cyclic hydrocarbon group, for example comprising from 1 to 20 carbon atoms, and further, for example, from 1 to 10 carbon atoms, and optionally comprising at least one group chosen from nitrogen, oxygen, and sulfur, and optionally substituted by at least one group chosen from —OR′, —COOR′, —COR′, —SH, —SR′, —OH, halogens, and a residue of a polymer which can be obtained by radical polymerization, by polycondensation or by ring opening, where R′ is a C₁-C₁₀ alkyl group.

According to at least one embodiment, R′3 is a saturated C₁-C₁₀ hydrocarbon group.

According to at least one embodiment, X is O.

Non-limiting mention may be made of the monomers of formula (I), of the monomers:
a) belonging to the family of the polyfluoroalkyl 2-cyanoacrylates, such as: the 2,2,3,3-tetrafluoropropyl ester of 2-cyano-2-propenoic acid of formula (II): 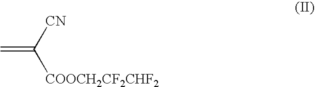
   or the 2,2,2-trifluoroethyl ester of 2-cyano-2-propenoic acid of formula (III): 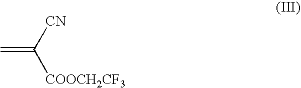
b) alkyl or alkoxyalkyl 2-cyanoacrylates of formula (IV): 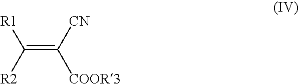
   wherein R′3 is chosen from C₁-C₁₀ alkyl, C₂-C₁₀ alkenyl and (C₁-C₄)alkoxy(C₁-C₁₀)alkyl radicals.

For example, non-limiting mention may be made of ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, methoxypropyl 2-cyanoacrylate, 2-octyl 2-cyanoacrylate, 2-propoxyethyl 2-cyanoacrylate, n-octyl 2-cyanoacrylate, allyl 2-cyanoacrylate, methoxypropyl 2-cyanoacrylate and isoamyl cyanoacrylate.

According to at least one embodiment, the monomers b) are used. For instance, the at least one cyanoacrylate monomer is chosen from C₆-C₁₀ alkyl cyanoacrylates.

The monomers that may be used, as disclosed herein, include but are not limited to the octyl cyanoacrylates of formula (V) and their mixtures: 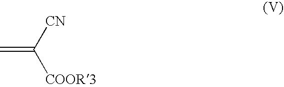
wherein:
R′3 is chosen from
   —(CH₂)₇—CH₃,
   —CH(CH₃)—(CH₂)₅—CH₃,
   —CH₂—CH(C₂H₅)—(CH₂)₃—CH₃,
   —(CH₂)₅—CH(CH₃)—CH₃, and
   —(CH₂)₄—CH(C₂H₅)—CH₃.

The cyanoacrylate monomer that may be used according to the present disclosure is methylheptyl 2-cyanoacrylate, sold under the commercial reference Ritelok CDN 1064 by Chemence.

The monomers used in accordance with the present disclosure can be covalently attached to at least one support, such as polymers, oligomers and dendrimers. The polymer or oligomer can be linear, branched, of comb structure or of block structure. The distribution of the monomers of the present disclosure over the polymer, oligomer or dendrimer structure can be random, in the end position or in the form of blocks.

As disclosed herein, the at least one cyanoacrylate monomers are monomers capable of polymerizing by the anionic route in the presence of a nucleophilic agent. As used herein, the term “anionic polymerization” is understood to mean the mechanism defined in the work “Advanced Organic Chemistry,” Third Edition, by Jerry March, pages 151 to 161.

The cyanoacrylate monomers of formula (I) according to the present disclosure can be synthesized according to the known methods described in the art. For instance, the cyanoacrylate monomers can be synthesized according to the teaching of U.S. Pat. No. 3,527,224; U.S. Pat. No. 3,591,767; U.S. Pat. No. 3,667,472; U.S. Pat. No. 3,995,641; U.S. Pat. No. 4,035,334 and U.S. Pat. No. 4,650,826.

The compositions employed in accordance with the present disclosure generally have a concentration of cyanoacrylate monomer according to the present disclosure ranging from 0.1 to 80% by weight, for instance from 1 to 50% by weight, with respect to the total weight of the composition, further for example, from 1 to 20% by weight, with respect to the total weight of the composition.

The nucleophilic agents capable of initiating anionic polymerization are systems known per se capable of generating a carbanion on contact with a nucleophilic agent, such as the hydroxide ions present in water. As used herein, the term “carbanion” is understood to mean the chemical entities defined in “Advanced Organic Chemistry,” Third Edition, by Jerry March, page 141.

The nucleophilic agents can be applied independent of the composition of the present disclosure. It can also be added to the composition of the present disclosure at the time of use.

The nucleophilic agent can be chosen from a molecular compound, an oligomer, a dendrimer and a polymer having nucleophilic functional groups. Non-limiting mention may be made, without implied limitation, as nucleophilic functional groups, of the functional groups: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ or H₂O, where Ph is a phenyl group, Ar is an aryl group, and R is a C₁-C₁₀ alkyl group.

The cyanoacrylate monomers can be synthesized according to the known methods described in the art. For instance, the at least one cyanoacrylate monomer can be synthesized according to the teaching of U.S. Pat. No. 3,527,224; U.S. Pat. No. 3,591,767; U.S. Pat. No. 3,667,472; U.S. Pat. No. 3,995,641; U.S. Pat. No. 4,035,334 and U.S. Pat. No. 4,650,826.

Polymerization inhibitors, for example anionic and/or radical polymerization inhibitors, can be introduced into the compositions, in order to increase the stability of the composition over time. Mention may be made, in a nonlimiting way, of the following polymerization inhibitors: sulfur dioxide, nitric oxide, boron trifluoride, hydroquinone and its derivatives, such as hydroquinone monoethyl ether or TBHQ, benzoquinone and its derivatives, such as duroquinone, catechol and its derivatives, such as t-butyl catechol and methoxycatechol, anisole and its derivatives, such as methoxyanisole or hydroxyanisole, pyrogallol and its derivatives, p-methoxyphenol, hydroxybutyltoluene, alkyl sulfates, alkyl sulfites, alkyl sulfones, alkyl sulfoxides, alkyl sulfides, mercaptans, 3-sulfonene and their mixtures. According to at least one embodiment, the alkyl groups have 1 to 6 carbon atoms.

Use may also be made, as inhibitor, of inorganic or organic acids.

Thus, the cosmetic composition according to the present disclosure can also comprise at least one inorganic or organic acid, the latter having at least one carboxyl or sulfo groups, exhibiting a pKa ranging from 0 to 6, such as phosphoric acid, hydrochloric acid, nitric acid, benzene- or toluenesulfonic acid, sulfuric acid, carbonic acid, hydrofluoric acid, acetic acid, formic acid, propionic acid, benzoic acid, mono-, di- or trichloroacetic acid, salicylic acid, trifluoroacetic acid, octanoic acid, heptanoic acid and hexanoic acid.

For example, according to at least one embodiment, acetic acid is used.

As disclosed herein, the concentration of inhibitor in the cosmetic composition may range from 10 ppm to 30% by weight, and further, for example from 10 ppm to 15% by weight, with respect to the total weight of the composition.

The term “glucide compound” is generally understood to mean at least one of the following compounds:
a monosaccharide;
an oligosaccharide; and
a polymer comprising at least one sugar unit.

The saccharide units can in addition be substituted by alkyl or hydroxyalkyl or alkoxy or acyloxy or carboxyl groups.

The term “monosaccharide” is understood to be the simplest nonhydrolyzable glucides. There are two types: aldoses, ketoses and sugar alcohols. They are also distinguished according to the number of carbon atoms which they have: trioses, pentoses, hexoses and ketoses. Non-limiting mention may be made, by way of example, of glucose, mannose, fructose, galactose, glucosamine, mannosamine, xylose, ribose, sorbitol, glucuronic acid and galacturonic acid.

The term “oligosaccharide” is understood to be a sequence of monosaccharides comprising from 2 to 10 monosaccharide units joined via glycoside bonds. Non-limiting mention may be made, by way of example, of lactose, sucrose, maltose, fructooligosaccharides and dextrins.

Polymer Comprising A Sugar Unit

The term “sugar unit” is generally understood, within the meaning of the present disclosure, as a monosaccharide portion, such as monosaccharide or glycoside or a simple sugar or an oligosaccharide portion, short chains formed by the linking of optionally different monosaccharide units or a polysaccharide portion, long chains composed of optionally different monosaccharide units, such as polysaccharides (homopolysaccharides or heteropolysaccharides).

According to the application as disclosed herein, the polymer comprising at least one sugar unit may be chosen from optionally modified natural polysaccharides and synthetic polymers comprising at least one glucide unit.

The polysaccharides may be chosen from fructans, gellans, glucans, modified or unmodified starches (such as those resulting, for example, from cereals, such as wheat, corn or rice, from vegetables, such as yellow split peas, or from tubers, such as potatoes or manioc), amylose, amylopectin, glycogen, dextrans, celluloses and their derivatives (methylcelluloses, hydroxyalkylcelluloses, ethylhydroxyethylcelluloses or carboxymethylcelluloses), mannans, xylans, lignins, arabans, galactans, galacturonans, chitin, chitosans, glucoronoxylans, arabinoxylans, xyloglucans, glucomannans, pectic acids and pectins, alginic acid and alginates, arabinogalactans, carrageenans, agars, glycosaminoglucans, gums arabic, gums tragacanth, ghatti gums, karaya gums, locust bean gums, galactomannans, such as guar gums and their nonionic derivatives (hydroxypropyl guar) and ionic derivatives, biopolysaccharide gums of microbial origin, such as scleroglucan or xanthan gums, mucopolysaccharides for instance chondroitin sulfates, and their mixtures.

As disclosed herein, the compounds of this type that may be used in the present disclosure are chosen from those which described, for example, in “Encyclopedia of Chemical Technology,” Kirk-Othmer, Third Edition, 1982, Vol. 3, pp. 896-900, and Vol. 15, pp. 439-458, in “Polymers in Nature,” by E. A. MacGregor and C. T. Greenwood, published by John Wiley & Sons, Chapter 6, pp. 240-328 (1980), in the work by Robert L. Davidson entitled “Handbook of Water-Soluble Gums and Resins,” published by McGraw Hill Book Company (1980), and in “Industrial Gums—Polysaccharides and their Derivatives,” edited by Roy L. Whistler, Second Edition, published by Academic Press Inc.,” the content of these four works being entirely included in the present disclosure by way of reference.

The nonionic guar gums may be modified or unmodified. The unmodified guar gums are, for example, the products sold under the name Vidogum GH 175 by Unipectine and under the names Meypro-Guar 50 and Jaguar C by Meyhall.

According to at least one embodiment, the modified nonionic guar gums are modified by C₁-C₆ hydroxyalkyl groups.

Further non-limiting mention may be made, among hydroxyalkyl groups, by way of example, of the hydroxymethyl, hydroxyethyl, hydroxypropyl and hydroxybutyl groups.

These guar gums are well-known in the state of the art and can, for example, be prepared by reacting the corresponding alkene oxides, such as, for example, propylene oxide, with the guar gum, so as to obtain a guar gum modified by hydroxypropyl groups. The degree of hydroxyalkylation, which corresponds to the number of molecules of alkylene oxide consumed per the number of free hydroxyl functional groups present on the guar gum, ranges from 0.4 to 1.2.

Such nonionic guar gums optionally modified by hydroxyalkyl groups are, for example, sold under the trade names Jaguar HP8, Jaguar HP60, Jaguar HP120, Jaguar DC 293 and Jaguar HP 105 by Rhône-Poulenc (Meyhall) or under the name Galactasol 4H4FD2 by Aqualon.

The anionic guar gums are polymers comprising groups derived from carboxylic, sulfonic or phosphoric acid.

The xanthan gums include, for example, the products sold under the names Keltrol, Keltrol T, Keltrol TF, Keltrol BT, Keltrol RD and Keltrol CG by Nutrasweet Kelco or under the names Rhodicare S and Rhodicare H by Rhodia Chimie.

The fructans or fructosans are oligosaccharides or polysaccharides comprising a sequence of anhydrofructose units optionally in combination with at least one saccharide residue other than fructose. The fructans may be linear or branched. The fructans include products obtained directly from a plant or microbial source or else products having a chain length which has been modified (increased or reduced) by fractionation, synthesis or hydrolysis, for instance enzymatically. The fructans generally have a degree of polymerization ranging from 2 to approximately 1000, for example from 3 to approximately 60.

Three groups of fructans are distinguished. The first group corresponds to products, the fructose units of which are for the most part bonded via β-2-1 bonds. These are essentially linear fructans, such as inulins. The second group also corresponds to linear fructoses but the fructose units are essentially bonded via β-2-6 bonds. These products are levans.

The third group corresponds to mixed fructans, that is to say having β-2-6 and β-2-1 sequences. These are essentially branched fructans, such as graminans. According to at least one embodiment, fructans are inulins. Inulin can be obtained, for example, from chicory, dahlias or Jerusalem artichokes.

According to at least one embodiment, the synthetic polymers comprising a sugar graft are chosen from:
polydimethylsiloxanes (PDMSs) comprising a sugar unit, which can be synthesized by reaction of a PDMS comprising an epoxy functional group with a carbohydrate, as described in the publication by R. Wagner et al., Appl. Organometallic Chem., 1998, Vol. 12, page 47, by reaction of an aldonolactone with an aminopropylsilicone or by hydrosilylation of an allyl glycoside with a polyhydromethylsiloxane (PHMS), as described in the publication by V. Braunmühl et al., Macromolecules, 1995, Vol. 28, page 17;
polymers comprising an acrylamide, acrylate, polyester, polyamide or vinyl backbone comprising a sugar unit, which can be synthesized by polyaddition, polycondensation or radical polymerization, as described in the publication by J. Kadokawa et al., Synlett., 1999, 1845-1856.

According to at least one embodiment, the polysaccharide is chosen from a cellulose, a hemicellulose, a lignocellulose, a starch, an inulin, a guar gum, a xanthan gum, a pullulan, an agar, a sodium, potassium or ammonium alginate, a carrageenan, a dextran, a furcellaran, a gellan gum, a gum arabic, a gum tragacanth, a hyaluronic acid, a konjac mannan, a lignosulfonate, a locust bean gum, a partially N-acylated chitin, a pectin, a polydextrose, a rhamsan gum and a welan gum. For instance, according to at least one embodiment, the polysaccharide is chosen from a cellulose, a hemicellulose, a carboxymethylcellulose, a hydroxyethylcellulose, a hydroxypropylcellulose, a hydroxypropylmethylcellulose, a methylcellulose, a lignocellulose, a starch, a starch acetate, a hydroxyethyl starch, a hydroxypropyl starch, an inulin, a guar gum, a carboxymethyl guar gum, a carboxymethyl hydroxypropyl guar gum, a hydroxyethyl guar gum, a hydroxypropyl guar gum and a xanthan gum, and their mixtures.

According to at least one embodiment, the polysaccharide has a weight-average molecular weight that ranges from 500 to 15,000,000, such as from 1,000 to 10,000,000.

The glucide compound or compounds are generally present in an amount that ranges from 0.01 to 80% by weight, such as from 0.1 to 40% by weight, with respect to the total weight of the composition, and further for example from 0.5 to 10% by weight.

As disclosed herein, the compositions may further comprise at least one pigment. This makes it possible to obtain a visible coloring over any type of background without leaching beforehand. This coloring is persistent toward shampooing operations.

The pigments of use in the present disclosure are chosen from all known organic and/or inorganic pigments of the art, such as those which are described in Kirk-Othmer's Encyclopedia of Chemical Technology and in Ullmann's Encyclopedia of Industrial Chemistry.

These pigments can be provided in the form of a powder or of a pigment paste. They can be coated or uncoated.

The pigments in accordance with the present disclosure may, for example, be chosen from white or colored pigments, lakes, special effect pigments, such as pearlescent agents or glitter, and their mixtures.

Non-limiting mention may be made, as examples of white or colored inorganic pigments, of titanium dioxide, which is or is not surface-treated, zirconium or cerium oxides, iron or chromium oxides, manganese violet, ultramarine blue, chromium hydrate and ferric blue. For example, as disclosed herein, the following inorganic pigments may be used: Ta₂O₅, Ti₃O₅, Ti₂O₃, TiO, ZrO₂ as a mixture with TiO₂, ZrO₂, Nb₂O₅, CeO₂, ZnS.

Non-limiting mention may be made, as examples of white or colored organic pigments, of nitroso, nitro, azo, xanthene, quinoline, anthraquinone or phthalocyanine compounds, compounds of metal complex type, or isoindolinone, isoindoline, quinacridone, perinone, perylene, diketopyrrolopyrrole, thioindigo, dioxazine, triphenylmethane and quinophthalone compounds.

For instance, white or colored organic pigments can be chosen from carmine, carbon black, aniline black, azo yellow, quinacridone, phthalocyanine blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, or the pigments obtained by oxidative polymerization of indole or phenol derivatives, as are described in French Patent No. FR 2 679 771.

Use may be made of pigment pastes formed of organic pigment, such as the products sold by Hoechst under the name:
Cosmenyl Yellow 1OG: Pigment Yellow 3 (CI 11710);
Cosmenyl Yellow G: Pigment Yellow 1 (CI 11680);
Cosmenyl Orange GR: Pigment Orange 43 (CI 71105);
Cosmenyl Red R: Pigment Red 4 (CI 12085);
Cosmenyl Carmine FB: Pigment Red 5 (CI 12490);
Cosmenyl Violet RL: Pigment Violet 23 (CI 51319);
Cosmenyl Blue A2R: Pigment Blue 15.1 (CI 74160);
Cosmenyl Green GG: Pigment Green 7 (CI 74260); and
Cosmenyl Black R: Pigment Black 7 (CI 77266).

The pigments in accordance with the present disclosure can also be in the form of composite pigments, as are described in European Patent No. EP 1 184 426. According to at least one embodiment, these composite pigments can be composed of particles comprising an inorganic core, at least one binder, which provides for the attachment of the organic pigments to the core, and at least one organic pigment which at least partially covers the core.

As used herein, the term “lake” is generally understood to mean dyes adsorbed onto insoluble particles, the combination thus obtained remaining insoluble during use. The inorganic substrates onto which the dyes are adsorbed are, for example, alumina, silica, calcium sodium borosilicate, calcium aluminum borosilicate and aluminum. Non-limiting mention may be made, among organic dyes, of cochineal carmine.

Non-limiting mention may be made, as examples of lakes, of the products known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 7 (CI 15 850:1), D & C Red 4 (CI 15 510), D & C Red 33 (CI 17 200), D & C Yellow 5 (CI 19 140), D & C Yellow 6 (CI 15 985), D & C Green (CI 61 570), D & C Yellow 1 O (CI 77 002), D & C Green 3 (CI 42 053) and D & C Blue 1 (CI 42 090).

As used herein, the term “special effect pigments” is understood to mean pigments which generally create a colored appearance (characterized by a certain hue, a certain vividness and a certain lightness) which is not uniform and which changes as a function of the conditions of observation (light, temperature, angles of observation, and the like). They thereby contrast with white or colored pigments, which provide a conventional opaque, semitransparent or transparent uniform color.

Non-limiting mention may be made, as examples of special effect pigments, of white pearlescent pigments, such as mica covered with titanium dioxide or with bismuth oxychloride, colored pearlescent pigments, such as mica covered with titanium dioxide and with iron oxides, mica covered with titanium dioxide, for instance with ferric blue or with chromium oxide or mica covered with titanium dioxide and with an organic pigment as defined above, and pearlescent pigments based on bismuth oxychloride. Non-limiting mention may be made, as pearlescent pigments, of the following pearlescent agents: Cellini sold by Engelhard (mica-TiO₂-lake), Prestige sold by Eckart (mica-TiO₂) or Colorona sold by Merck (mica-TiO₂-Fe₂O₃).

Non-limiting mention may also be made of pigments with an interference effect which are not attached to a substrate, such as liquid crystals (Helicones HC from Wacker), interference holographic glitter (Geometric Pigment or Spectra f/x from Spectratek). Special effect pigments may also comprise fluorescent pigments, whether substances which are fluorescent in daylight or which produce ultraviolet fluorescence, phosphorescent pigments, photochromic pigments, thermochromic pigments and quantum dots, for example sold by Quantum Dots Corporation.

Quantum dots are luminescent semiconductor nanoparticles capable of emitting, under light excitation, radiation exhibiting a wavelength ranging from 400 nm to 700 nm. These nanoparticles are known from the literature. For instance, they can be manufactured according to the processes described, for example, in U.S. Pat. No. 6,225,198 or U.S. Pat. No. 5,990,479, in the publications which are cited therein and in the following publications: Dabboussi B. O. et al., “(CdSe)ZnS core-shell quantum dots: synthesis and characterisation of a size series of highly luminescent nanocrystallites”, Journal of Physical Chemistry B, Vol.101, 1997, pp. 9463-9475, and Peng, Xiaogang et al., “Epitaxial growth of highly luminescent CdSe/CdS core/shell nanocrystals with photostability and electronic accessibility,” Journal of the American Chemical Society, Vol. 119, No. 30, pp. 7019-7029.

The variety of the pigments which can be used in the present disclosure makes it possible to obtain a rich palette of colors as well as specific optical effects, such as interference, metallic effects.

According to at least one embodiment, the pigments are colored pigments. As disclosed herein, the term “colored pigments” is generally understood to mean pigments other than white pigments.

The size of the pigment of use in the context of the present disclosure generally range from 10 nm to 200 μm, such as from 20 nm to 80 μm and further, for example from 30 nm to 50 μm.

The pigment or pigments are each generally present in the composition in accordance with the present disclosure in amounts generally ranging from 0.05 to 50% of the total weight of the composition, for example from 0.1 to 35%.

The compositions according to the present disclosure may comprise a gelling/structuring agent for nonaqueous media, such as the PSPA silicone polyamides (DP100 and DP15) sold by Dow Chemical, the polylauryldimethylsiloxane organic KSG products and the silicone KSG products sold by Shinetsu, dextrin palmitate and inulin stearate (Rheopearl range from Chiba Flour Milling), the acrylates comprising an alkyl chain sold by Landec, the ethylene/octene copolymers sold by Dupont de Nemours, the dibutyl lauroyl glutamide sold by Ajinomoto, disorbene sold by Roquette, the styrene/acrylate copolymers Versagel M5960 and 5670 sold by Penreco, the diblock and triblock Kraton products sold by Kraton Polymers, hydroxystearic acid, jojoba waxes, the pyrogenic silicas sold by Degussa, the silicone waxes sold by Waker, the polyamide Uniclear sold by Arizona Chemical, or bentone.

The compositions according to the present disclosure may further comprise a propellant. The propellant may comprise compressed or liquefied gases commonly employed in the preparation of aerosol compositions. According to at least one embodiment, air, carbon dioxide, compressed nitrogen or alternatively a soluble gas, such as dimethyl ether, halogenated (for instance, fluorinated) and nonhalogenated hydrocarbons (butane, propane, isobutane), and their mixtures may be used.

The compositions in accordance with the present disclosure may also comprise at least one adjuvant commonly used in cosmetics chosen, for example, from reducing agents, fatty substances, plasticizers, softening agents, antifoaming agents, moisturizing agents, pigments, clays, inorganic fillers, UV screening agents, colloidal minerals, peptizing agents, solubilizing agents, fragrances, preservatives, anionic, cationic, nonionic or amphoteric surfactants, fixing or nonfixing polymers, nonglucoside polyols, proteins, vitamins, direct or oxidation dyes, pearlescent agents, propellants, and inorganic or organic thickening agents and N-acylamino acids, oxyethylenated or nonoxyethylenated waxes, paraffins, C₁₀-C₃₀ fatty acids, such as stearic acid or lauric acid, C₁₀-C₃₀ fatty amides, such as lauric acid diethanolamide, solid esters of C₁₀-C₃₀ fatty alcohol, and their mixtures.

The at least one adjuvant or the at least one glucide compound can optionally be encapsulated. The capsule can be of polycyanoacrylate type.

For instance, the composition may be used on keratinous fibers, such as the hair, for example in the presence of a nucleophilic agent, for their cosmetic treatment.

Another aspect of the present disclosure relates to a method for conferring a conditioning effect, such as softness, disentangling, gloss or volume, and/or a styling effect, such as the form retention of the hair.

A cosmetic treatment process according to the present disclosure comprises the application of a composition as defined above to keratinous fibers, such as the hair, and according to one embodiment, the process may occur in the presence of at least one nucleophilic agent as defined below.

The at least one nucleophilic agent capable of initiating anionic polymerization are systems known per se capable of generating a carbanion on contact with a nucleophilic agent, such as the hydroxide ions present in water at neutral pH. The term “carbanion” is generally understood to mean the chemical entities defined in “Advanced Organic Chemistry,” Third Edition, by Jerry March, page 141.

The nucleophilic agents may be composed of a molecular compound, an oligomer, a dendrimer and a polymer having nucleophilic functional groups. Non-limiting mention may be made, without implied limitation, as nucleophilic functional groups, of the functional groups: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, where Ph is a phenyl group, Ar is an aryl group, and R is a C₁-C₁₀ alkyl group.

According to at least one embodiment, the at least one nucleophilic agent is water. This water may be applied with a preliminary moistening of the keratin fibers.

It is also possible, in order to adjust the reaction kinetics, to moisten keratinous fibers, such as the hair, beforehand using an aqueous solution, the pH of which has been adjusted using a base, an acid or an acid/base mixture. The acid and/or the base can be inorganic or organic.

It is also possible to adjust the kinetics of polymerization by the anionic route by preimpregnating keratinous fibers, such as the hair, using a nucleophilic agent other than water. The at least one nucleophilic agent can be used pure, in solution or in the form of an emulsion or can be encapsulated.

In order to adjust the kinetics of anionic polymerization, it is also possible to increase the nucleophilicity of keratinous fibers, such as the hair, by chemical conversion of the keratinous substance.

Non-limiting mention may be made, by way of example of chemical conversion, of the reduction of the disulfide bridges of which the keratin is partially composed to give thiols before application of the composition of the present disclosure. Further non-limiting mention may be made, nonexhaustively, as reducing agents for the disulfide bridges of which the keratin is partially composed, of the following compounds:
anhydrous sodium thiosulfate, powdered sodium metabisulfite, thiourea, ammonium sulfite, thioglycolic acid, thiolactic acid, ammonium thiolactate, glyceryl monothioglycolate, ammonium thioglycolate, thioglycerol, 2,5-dihydroxybenzoic acid, diammonium dithioglycolate, strontium thioglycolate, calcium thioglycolate, zinc formaldehyde sulfoxylate, isooctyl thioglycolate, d,l-cysteine and monoethanolamine thioglycolate.

In order to adjust the kinetics for polymerization by the anionic route, for instance to reduce the rate of polymerization of the monomers of the present disclosure, it is possible to increase the viscosity of the composition. To do this, at least one polymer which does not react with the monomers in accordance with the present disclosure can be added to the disclosed composition. In this context, non-limiting mention may be made, nonexhaustively, of poly(methyl methacrylate) (PMMA) or cyanoacrylate-based copolymers, such as are described in U.S. Pat. No. 6,224,622.

In order to improve, inter alia, the adhesion of the poly(cyanoacrylate) formed in situ, the fiber can be pretreated with all types of polymers or a hair treatment can be carried out before application of the composition of the present disclosure, for example a direct or oxidation dyeing, a perming or a hair straightening.

The application of the compositions as defined above may or may not be followed by a rinsing operation.

They can be provided in the form of a lotion, spray or foam.

The composition in accordance with the present disclosure can be provided as is or can result from the mixing at the time of use of two compositions. For example, the first composition can comprise the at least one cyanoacrylate monomer and the second composition can comprise the at least one glucide compound.

According to at least one embodiment, the composition comprising the at least one cyanoacrylate monomer is anhydrous. The composition comprising the glucide compound or compounds can be anhydrous, aqueous or alcoholic and can optionally be provided in the form of water-in-oil or oil-in-water emulsions. When the glucide compound is provided in the form of aqueous or alcoholic (for example, ethanolic) solutions or dispersions, the composition comprising it is generally aqueous or alcoholic in the form of water-in-oil or oil-in-water emulsions.

The process according to the present disclosure comprises the application to keratinous fibers of a composition comprising, in a cosmetically acceptable medium, at least one glucide compound and at least one cyanoacrylate monomer as defined above in the presence of at least one nucleophilic agent.

According to an alternative form of the process of the present disclosure, the application of the composition in accordance with the present disclosure is carried out in at least two stages, wherein one of the stages comprises applying, to keratinous fibers, a composition comprising the glucide compound or compounds, with or without intermediate rinsing, and another stage comprises applying, to the keratinous fibers, a composition comprising the at least one cyanoacrylate monomer. The order of these stages is irrelevant.

One embodiment of the present disclosure comprises a process comprising applying the at least one cyanoacrylate monomer prior to applying the at least one glucide compound.

Another embodiment of the present disclosure comprises a process comprising applying the at least one glucide compound prior to applying the at least one cyanoacrylate monomer.

A further embodiment of the present disclosure comprises a process comprising the following stages:
(1) applying, to the hair, a composition comprising from 0.1 to 59.9% by weight and further, for example from 0.25 to 25% by weight of a glucide compound, the percentages being expressed with respect to the total weight of the solution or of the dispersion,
(2) applying, to the hair, after an optional intermediate rinsing operation, a composition comprising from 0.1 to 40% by weight, for example from 1 to 20% by weight, of at least one cyanoacrylate monomer as defined above, the percentages being expressed with respect to the total weight of the composition.

The order of the two stages (1) and (2) can be inverted.

The application of a cosmetic product can in addition precede the first stage or follow the second stage. Each stage can also be interrupted by a rinsing operation and optionally by a drying operation, it being possible for the drying operation to be carried out using a hood dryer, a hairdryer, a flat iron and/or curling tongs.

The stages of the processes described above can be repeated so as to obtain several layers, depending on the type of coating which is desired in terms of chemical nature, mechanical strength, thickness, appearance and/or feel.

A further aspect of the present disclosure is a kit comprising a first composition comprising at least one cyanoacrylate monomer as defined above and optionally at least one anionic and/or radical polymerization inhibitor as defined above, as well as a second composition comprising, in a cosmetically acceptable medium, at least one glucide compound and optionally a third composition which comprises the at least one nucleophilic agent.

Another aspect of the present disclosure is a kit for the treatment of keratinous fibers, such as the hair, comprising a first composition comprising at least one glucide compound as defined above and a second composition comprising at least one cyanoacrylate monomer as defined above and optionally at least one polymerization inhibitor.

Another aspect of the present disclosure relates to a kit comprising a first composition comprising at least one cyanoacrylate monomer as defined above and a liquid organic solvent and optionally at least one anionic and/or radical polymerization inhibitor and a second composition comprising, in a cosmetically acceptable medium, at least one glucide compound.

According to at least one embodiment of the kit as disclosed herein, the composition comprises the at least one glucide compound and further comprises at least one nucleophilic agent as defined above.

According to another embodiment of the present disclosure, the kit further comprises a composition comprising at least one nucleophilic agent as defined above.

According to a first embodiment of the disclosed kit, the composition comprising the at least one glucide compound and the composition comprising the at least one cyanoacrylate monomer and optionally the at least one polymerization inhibitor are present in a single anhydrous composition.

According to a second alternative form, the composition comprising the at least one glucide compound is an aqueous composition and the composition comprising the at least one cyanoacrylate monomer and optionally the at least one polymerization inhibitor is an anhydrous composition. In this case, the compositions are mixed at the time of use or are applied successively.

The at least one cyanoacrylate monomer is present in the composition comprising them in an amount generally ranging from 5 to 80% by weight approximately of the total weight of the composition, such as from 1 to 30% by weight and further for example from 0.1 to 20% by weight.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in its respective testing measurement.

In the following examples, all the amounts are shown as percent by weight of active material with respect to the total weight of the composition, unless otherwise indicated.

The examples that follow are intended to illustrate the present disclosure without, however, being limiting in nature.

### EXAMPLES

### Example 1

The following composition A was prepared:

| | |
|---|---|
| Hydroxyethylcellulose | 1.5 g |
| Water | 98.5 g |

The following composition B was prepared:

| | | |
|---|---|---|
| α,ω-Dihydroxylated polydimethylsiloxane/ | 45 | g |
| cyclopentadimethylsiloxane (14.7/85.3% by weight), | | |
| sold by Dow Corning under the name of DC 1501 Fluid | | |
| Cyclopentadimethylsiloxane, sold by Dow Corning under | 44.75 | g |
| the name of DC245 Fluid | | |
| Acetic acid | 0.25 | g |
| Methylheptyl cyanoacrylate, from Chemence | 10 | g |

0.3 g of the composition A was applied to a lock of hair weighing 1 g shampooed beforehand. Subsequently, 0.5 g of the composition B was applied. After leaving at ambient temperature for 15 min, the lock was dried with a hairdryer for 3 min.

The hair was sheathed and texturized to the touch. This effect was still visible after several shampooing operations.

The hair did not become greasy over time, in contrast to the hair treated solely with water instead of the composition A. The hair had a clean appearance.

### Example 2

The following composition A was prepared:

| | | |
|---|---|---|
| Carboxymethyl hydroxypropyl guar | 1.5 | g |
| Water | 98.5 | g |

The following composition B was prepared:

| | | |
|---|---|---|
| α,ω-Dihydroxylated polydimethylsiloxane/ | 45 | g |
| cyclopentadimethylsiloxane (14.7%/85.3% by weight), | | |
| sold by Dow Corning under the name of DC 1501 Fluid | | |
| Cyclopentadimethylsiloxane, sold by Dow Corning under | 44.75 | g |
| the name of DC245 Fluid | | |
| Acetic acid | 0.25 | g |
| Methylheptyl cyanoacrylate, from Chemence | 10 | g |

0.3 g of the composition A was applied to a lock of hair weighing 1 g washed beforehand with a shampoo. Subsequently, 0.5 g of the composition B was applied. After leaving at ambient temperature for 15 min, the lock was dried with a hairdryer for 3 min.

The hair was sheathed and texturized to the touch. This effect was still visible after several shampooing operations.

The hair did not become greasy over time, in contrast to hair treated solely with water instead of the composition A. The hair had a clean appearance.

### Example 3

The following composition A was prepared:

| | A | |
|---|---|---|
| Sodium chondroitin sulfate* | 1.5 | g |
| Water | 98.5 | g |

*Sold by Sederma under the name of Hydromarine

The following composition B was prepared:

| | | |
|---|---|---|
| α,ω-Dihydroxylated polydimethylsiloxane/ | 45 | g |
| cyclopentadimethylsiloxane (14.7/85.3), sold by Dow | | |
| Corning under the name of DC 1501 Fluid | | |
| Cyclopentadimethylsiloxane, sold by Dow Corning under | 44.75 | g |
| the name of DC245 Fluid | | |
| Acetic acid | 0.25 | g |
| Methylheptyl cyanoacrylate, from Chemence | 10 | g |

0.3 g of the composition A was applied to a lock of hair weighing 1 g washed beforehand with a shampoo. Subsequently, 0.5 g of the composition B was applied. After leaving at ambient temperature for 15 min, the lock was dried with a hairdryer for 3 min.

The hair was sheathed and texturized to the touch. This effect was still visible after several shampooing operations.

The hair did not become greasy over time, in contrast to hair treated solely with water instead of the composition A. The hair had a clean appearance.

What is claimed is:

1. A cosmetic composition comprising:
at least one cyanoacrylate monomer;
at least one glucide compound; and
at least one liquid organic solvent,
wherein the at least one cyanoacrylate monomer is chosen from the monomers of formula (I): 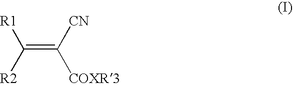
wherein:
X is chosen from NH, S and O;
R1 and R2 are each independently hydrogen atoms; and
R′3 is chosen from a hydrogen atom, a saturated, unsaturated, linear, branched or cyclic hydrocarbon group, comprising from 1 to 20 carbon atoms and optionally comprising at least one group chosen from nitrogen, oxygen, and sulfur, and optionally substituted by at least one group chosen from —OR′, —COOR′, —COR′, —SH, —SR′, —OH, halogens, and a residue of a polymer which can be obtained by radical polymerization, by polycondensation or by ring opening, where R′ is a C₁-C₁₀ alkyl group.

2. The cosmetic composition according to claim 1, wherein the at least one cyanoacrylate monomer is chosen from the monomers of formula (IV): 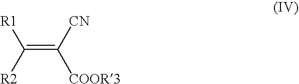
wherein:
R1 and R2 are each independently hydrogen atoms; and
R′3 is chosen from C₁-C₁₀ alkyl, C₂-C₁₀ alkenyl and (C₁-C₄)alkoxy(C₁-C₁₀)alkyl radicals.

3. The cosmetic composition according to claim 2, wherein the at least one cyanoacrylate monomer is chosen from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, methoxypropyl 2-cyanoacrylate, 2-octyl 2-cyanoacrylate, 2-propoxyethyl 2-cyanoacrylate, n-octyl 2-cyanoacrylate, allyl 2-cyanoacrylate, methoxypropyl 2-cyanoacrylate and isoamyl cyanoacrylate.

4. The cosmetic composition according to claim 3, wherein the at least one cyanoacrylate monomer is chosen from C₆-C₁₀ alkyl cyanoacrylates.

5. The cosmetic composition according to claim 4, wherein the at least one cyanoacrylate monomer is chosen from the octyl cyanoacrylate monomers of formula (V) and their mixtures: 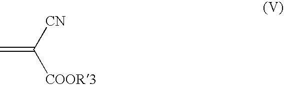
wherein:
R′3 is chosen from
—(CH₂)₇—CH₃,
—CH(CH₃)—(CH₂)₅—CH₃,
—CH₂—CH(C₂H₅)—(CH₂)₃—CH₃,
—(CH₂)₅—CH(CH₃)—CH₃, and
—(CH₂)₄—CH(C₂H₅)—CH₃.

6. The cosmetic composition according to claim 1, wherein the at least one cyanoacrylate monomer is covalently attached to at least one support chosen from polymers, oligomers, and dendrimers.

7. The cosmetic composition according to claim 1, wherein the at least one cyanoacrylate monomer is present in an amount ranging from 0.1 to 80% by weight, with respect to the total weight of the composition.

8. The cosmetic composition according to claim 1, wherein the at least one glucide compound is chosen from:
monosaccharides;
oligosaccharides; and
polymers comprising at least one sugar unit.

9. The cosmetic composition according to claim 1, wherein the at least one glucide compound is chosen from glucose, mannose, fructose, galactose, glucosamine, mannosamine, xylose, ribose, sorbitol, glucuronic acid, and galacturonic acid.

10. The cosmetic composition according to claim 1, wherein the at least one glucide compound is chosen from lactose, sucrose, maltose, fructooligosaccharides, and dextrins.

11. The cosmetic composition according to claim 8, wherein the polymers comprising at least one sugar unit are chosen from optionally modified natural polysaccharides and synthetic polymers comprising at least one glucide unit.

12. The cosmetic composition according to claim 11, wherein the polysaccharides are chosen from fructans, gellans, glucans, modified or unmodified starches, amylose, amylopectin, glycogen, dextrans, celluloses and their derivatives, mannans, xylans, lignins, arabans, galactans, galacturonans, chitin, chitosans, glucoronoxylans, arabinoxylans, xyloglucans, glucomannans, pectic acids and pectins, alginic acid and alginates, arabinogalactans, carrageenans, agars, glycosaminoglucans, gums arabic, gums tragacanth, ghatti gums, karaya gums, locust bean gums, galactomannans, biopolysaccharide gums of microbial origin, mucopolysaccharides, and their mixtures.

13. The cosmetic composition according to claim 12, wherein the polysaccharides are chosen from a cellulose, a hemicellulose, a carboxymethylcellulose, a hydroxyethylcellulose, a hydroxypropylcellulose, a hydroxypropylmethylcellulose, a methylcellulose, a lignocellulose, a starch, a starch acetate, a hydroxyethyl starch, a hydroxypropyl starch, an inulin, a guar gum, a carboxymethyl guar gum, a carboxymethyl hydroxypropyl guar gum, a hydroxyethyl guar gum, a hydroxypropyl guar gum, a xanthan gum, and their mixtures.

14. The cosmetic composition according to claim 1, wherein the at least one glucide compound is present in an amount ranging from 0.001% to 80% by weight with respect to the total weight of the final composition.

15. The cosmetic composition according to claim 14, wherein the at least one glucide compound is present in an amount ranging from 0.01% to 50% by weight with respect to the total weight of the final composition.

16. The cosmetic composition according to claim 1, wherein the composition is anhydrous.

17. The cosmetic composition according to claim 1, wherein the at least one liquid organic solvent is chosen from aromatic alcohols; liquid fatty alcohols; modified or unmodified polyols; volatile silicones; liquid modified polydimethylsiloxanes; mineral, organic or vegetable oils; alkanes; liquid fatty acids; liquid fatty esters; and their mixtures.

18. The cosmetic composition according to claim 1, wherein the at least one liquid organic solvent is present in an amount ranging from 0.01 to 99% by weight with respect to the total weight of the composition.

19. The cosmetic composition according to claim 18, wherein the at least one liquid organic solvent is present in an amount ranging from 0.1 to 50% by weight with respect to the total weight of the composition.

20. The cosmetic composition according to claim 1, further comprising at least one polymerization inhibitor.

21. The cosmetic composition according to claim 20, wherein the at least one polymerization inhibitor is an anionic and/or radical polymerization inhibitor.

22. The cosmetic composition according to claim 20, wherein the at least one polymerization inhibitor is chosen from sulfur dioxide, nitric oxide, lactone, boron trifluoride, hydroquinone and its derivatives, benzoquinone and its derivatives, catechol and its derivatives, anisole and its derivatives, hydroxyanisole or butylhydroxyanisole, pyrogallol, 2,4-dinitrophenol, 2,4,6-trihydroxybenzene, p-methoxyphenol, hydroxybutyltoluene, alkyl sulfates, alkyl sulfites, alkyl sulfones, alkyl sulfoxides, alkyl sulfides, mercaptans, 3-sulfonene, inorganic and organic acids, and their mixtures.

23. The cosmetic composition according to claim 20, wherein the at least one polymerization inhibitor is present in an amount ranging from 10 ppm to 20%, with respect to the total weight of the composition.

24. The cosmetic composition according to claim 1, which additionally comprises at least one adjuvant chosen from reducing agents, fatty substances, plasticizers, softening agents, antifoaming agents, moisturizing agents, pigments, clays, inorganic fillers, UV screening agents, colloidal minerals, peptizing agents, solubilizing agents, fragrances, preservatives, anionic, cationic, nonionic or amphoteric surfactants, fixing or nonfixing polymers, nonglucosidic polyols, proteins, vitamins, direct or oxidation dyes, pearlescent agents, propellants, and inorganic or organic thickening agents, oxyethylenated or nonoxyethylenated waxes, paraffins, C₁₀-C₃₀ fatty acids, C₁₀-C₃₀ fatty amides, solid esters of C₁₀-C₃₀ fatty alcohol, and their mixtures.

25. A method for the cosmetic treatment and/or care of keratinous fibers comprising:
applying a cosmetic composition to the keratinous fibers;
where the cosmetic composition comprises:
at least one cyanoacrylate monomer;
at least one glucide compound; and
at least one liquid organic solvent,
wherein the at least one cyanoacrylate monomer is chosen from the monomers of formula (I): 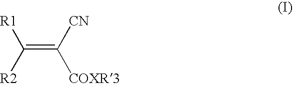
wherein:
X is chosen from NH, S and O;
R1 and R2 are each independently hydrogen atoms; and
R′3 is chosen from a hydrogen atom, a saturated, unsaturated, linear, branched or cyclic hydrocarbon group, comprising from 1 to 20 carbon atoms and optionally comprising at least one group chosen from nitrogen, oxygen, and sulfur, and optionally substituted by at least one group chosen from —OR′, —COOR′, —COR′, —SH, —SR′, —OH, halogens, and a residue of a polymer which can be obtained by radical polymerization, by polycondensation or by ring opening, where R′ is a C₁-C₁₀ alkyl group.

26. The method according to claim 25, which occurs in the presence of a nucleophilic agent.

27. The method as claimed in claim 26, wherein the nucleophilic agent is chosen from molecular compounds, oligomers, dendrimers and polymers having nucleophilic functional groups chosen from: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, where Ph is a phenyl group, Ar is an aryl group, and R is a C₁-C₁₀ alkyl group.

28. The method as claimed in claim 27, wherein the nucleophilic agent is water.

29. A method for conferring softness, disentangling, gloss, volume and/or form retention on the hair comprising:
applying a cosmetic composition to the hair;
where the cosmetic composition comprises:
at least one cyanoacrylate monomer;
at least one glucide compound; and
at least one liquid organic solvent,
wherein the at least one cyanoacrylate monomer is chosen from the monomers of formula (I): 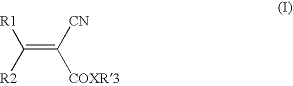
wherein:
X is chosen from NH, S and O;
R1 and R2 are each independently hydrogen atoms; and
R′3 is chosen from a hydrogen atom, a saturated, unsaturated, linear, branched or cyclic hydrocarbon group, comprising from 1 to 20 carbon atoms and optionally comprising at least one group chosen from nitrogen, oxygen, and sulfur, and optionally substituted by at least one group chosen from —OR′, —COOR′, —COR′, —SH, —SR′, —OH, halogens, and a residue of a polymer which can be obtained by radical polymerization, by polycondensation or by ring opening, where R′ is a C₁-C₁₀ alkyl group.

30. The method according to claim 29, which occurs in the presence of a nucleophilic agent.

31. The method according to claim 30, wherein the nucleophilic agent is chosen from molecular compounds, oligomers, dendrimers and polymers having nucleophilic functional groups chosen from: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, where Ph is a phenyl group, Ar is an aryl group, and R is a C₁-C₁₀ alkyl group.

32. The method according to claim 31, wherein the nucleophilic agent is water.

33. A process for the cosmetic treatment of keratinous fibers comprising:
applying a cosmetic composition to keratinous fibers in the presence of a nucleophilic agent,
wherein the cosmetic composition comprises:
at least one cyanoacrylate monomer;
at least one glucide compound; and
at least one liquid organic solvent,
wherein the at least one cyanoacrylate monomer is chosen from the monomers of formula (I): 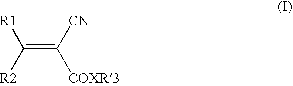
wherein:
X is chosen from NH, S and O;
R1 and R2 are each independently hydrogen atoms; and
R′3 is chosen from a hydrogen atom, a saturated, unsaturated, linear, branched or cyclic hydrocarbon group, comprising from 1 to 20 carbon atoms and optionally comprising at least one group chosen from nitrogen, oxygen, and sulfur, and optionally substituted by at least one group chosen from —OR′, —COOR′, —COR′, —SH, —SR′, —OH, halogens, and a residue of a polymer which can be obtained by radical polymerization, by polycondensation or by ring opening, where R′ is a C₁-C₁₀ alkyl group.

34. The process according to claim 33, wherein the nucleophilic agent is chosen from molecular compounds, oligomers, dendrimers, and polymers having nucleophilic functional groups chosen from: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, where Ph is a phenyl group, Ar is an aryl group, and R is a C₁-C₁₀ alkyl group.

35. The process according to claim 34, wherein the nucleophilic agent is water.

36. The process according to claim 33, wherein the composition is applied to keratinous fibers moistened beforehand using an aqueous solution, the pH of which has been adjusted using a base, an acid or an acid/base mixture.

37. The process according to claim 33, wherein the keratinous fibers are preimpregnated using a nucleophilic agent other than water.

38. The process according to claim 33, wherein the keratinous fibers are reduced beforehand, prior to applying the cosmetic composition.

39. The process according to claim 33, wherein the application of the composition is followed by a rinsing operation.

40. The process according to claim 33, wherein the keratinous fibers are the hair.

41. A process for the cosmetic treatment of the hair comprising at least two stages, wherein:
a first stage comprises applying at least one glucide compound and at least one liquid organic solvent, with or without intermediate rinsing; and
a second stage comprises applying at least one cyanoacrylate monomer, wherein the at least one cyanoacrylate monomer is chosen from the monomers of formula (I): 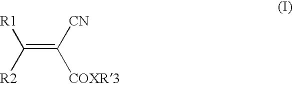
wherein:
X is chosen from NH, S and O;
R1 and R2 are each independently hydrogen atoms; and
R′3 is chosen from a hydrogen atom, a saturated, unsaturated, linear, branched or cyclic hydrocarbon group, comprising from 1 to 20 carbon atoms and optionally comprising at least one group chosen from nitrogen, oxygen, and sulfur, and optionally substituted by at least one group chosen from —OR′, —COOR′, —COR′, —SH, —SR′, —OH, halogens, and a residue of a polymer which can be obtained by radical polymerization, by polycondensation or by ring opening, where R′ is a C₁-C₁₀ alkyl group.

42. The process according to claim 41, wherein the application of at least one glucide compound occurs prior to the application of said at least one cyanoacrylate monomer.

43. The process according to claim 41, wherein the application of said at least one cyanoacrylate monomer occurs prior to the application of at least one glucide compound.

44. The process according to claim 41, comprising the stages:
(1) applying, to the hair, a composition comprising from 0.1 to 59.9% by weight of a glucide compound;
(2) applying, to the hair, after an optional intermediate rinsing operation, a composition comprising from 0.1 to 40% by weight, of said at least one cyanoacrylate monomer.

45. A kit comprising:
a first composition comprising at least one cyanoacrylate monomer and optionally at least one anionic and/or radical polymerization inhibitor,
wherein the at least one cyanoacrylate monomer is chosen from the monomers of formula (I): 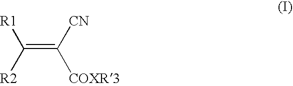
X is chosen from NH, S and O;
R1 and R2 are each independently hydrogen atoms; and
R′3 is chosen from a hydrogen atom, a saturated, unsaturated, linear, branched or cyclic hydrocarbon group, comprising from 1 to 20 carbon atoms and optionally comprising at least one group chosen from nitrogen, oxygen, and sulfur, and optionally substituted by at least one group chosen from —OR′, —COOR′, —COR′, —SH, —SR′, —OH, halogens, and a residue of a polymer which can be obtained by radical polymerization, by polycondensation or by ring opening, where R′ is a C₁-C₁₀ alkyl group;
a second composition comprising an organic solvent; and
a third composition comprising, in a cosmetically acceptable medium, at least one glucide compound.

46. A kit comprising:
a first composition comprising at least one cyanoacrylate monomer and a liquid organic solvent and optionally at least one anionic and/or radical polymerization inhibitor,
wherein the at least one cyanoacrylate monomer is chosen from the monomers of formula (I): 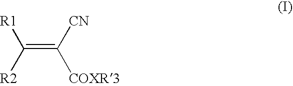
wherein:
X is chosen from NH, S and O;
R1 and R2 are each independently hydrogen atoms; and
R′3 is chosen from a hydrogen atom, a saturated, unsaturated, linear, branched or cyclic hydrocarbon group, comprising from 1 to 20 carbon atoms and optionally comprising at least one group chosen from nitrogen, oxygen and sulfur, and optionally substituted by at least one group chosen from —OR′, —COOR′, —COR′, —SH, —SR′, —OH, halogens, and a residue of a polymer which can be obtained by radical polymerization, by polycondensation or by ring opening, where R′ is a C₁-C₁₀ alkyl group; and
a second composition comprising, in a cosmetically acceptable medium, at least one glucide compound.
